(19) United States
(12) Patent Application Publication
(10) Pub. No.: US 2023/0226315 A1
(43) Pub. Date: Jul. 20, 2023

(54) VASCULAR ACCESS SYSTEM

(71) Applicant: ABIOMED, Inc., Danvers, MA (US)

(72) Inventors: Emilia Jahangir, Danvers, MA (US); Geoffrey Christanday, Danvers, MA (US); Zachary Nickerson, Danvers, MA (US); Brian Chouinard, Danvers, MA (US); David Schoenberg, Danvers, MA (US)

(73) Assignee: ABIOMED, Inc., Danvers, MA (US)

(21) Appl. No.: 18/071,765

(22) Filed: Nov. 30, 2022

Related U.S. Application Data
(60) Provisional application No. 63/285,300, filed on Dec. 2, 2021.

(51) Int. Cl. A61M 25/01; A61M 39/02; A61M 60/13; A61M 25/00

(52) U.S. Cl. CPC A61M 25/0113; A61M 39/0247; A61M 60/13; A61M 25/0023

(57) ABSTRACT

A vascular access system comprising a sheath, a slitted sleeve, and a sealing portion of the slitted sleeve. The slitted sleeve comprises a tubular sleeve body having a longitudinal slit along its length, wherein the slitted sleeve is adapted to receive a first portion of a medical device therein. The longitudinal slit closes, but does not seal, after the first portion of the medical device is inserted therein. The sealing portion of the slitted sleeve is configured to fill a gap and form a seal between an inner surface of the sheath and a portion of the first portion of the medical device disposed in the sheath when the sheath is inserted in the blood vessel and the slitted sleeve is inserted into the sheath lumen. A vascular access device accessory and a method for inserting it into a sheath are also provided.

### CROSS-REFERENCE TO RELATED APPLICATIONS

This application claims the benefit of U.S. Provisional Application No. 63/285,300, filed Dec. 2, 2021, the disclosure of which is incorporated by reference herein in its entirety.

### BACKGROUND

A medical device, such as an intracardiac heart pump assembly, can be introduced into a patient in various ways. In general, a heart pump can be introduced in the heart to pump blood from the heart into a vessel to support the function of the heart. When deployed in the heart, a heart pump assembly pulls blood from the left ventricle of the heart and expels blood into the aorta, or pulls blood from the inferior vena cava (IVC), bypasses the right atrium and right ventricle, and expels blood into the pulmonary artery. Heart pump assemblies are introduced surgically or percutaneously during a cardiac procedure through the vascular system. In one common approach, pump assemblies are inserted by a catheterization procedure through the femoral artery using a sheath. The sheath may alternatively be inserted in other locations such as in the femoral vein or any path for delivery of a pump for supporting either the left or right side of the heart.

Generally, the introducer sheath may be inserted into the femoral artery through an arteriotomy to create an insertion path for the pump assembly. A portion of the pump assembly is then advanced through an inner lumen of the introducer and into the artery. Some medical introducers have expandable sheath bodies which may expand radially to allow passage of percutaneous devices, such as a heart pump assembly, into the patient's vasculature. Such an introducer is inserted having a resting inner diameter smaller than the outer diameter of the widest portion of the percutaneous device being introduced. The introducer expands to allow passage of the percutaneous device through the sheath and into the vasculature and then shrinks again to its resting inner diameter after the widest portion of the device has passed through the introducer.

The resting inner diameter of the introducer, however, is greater than that of one or more of the narrower portion(s) of the pump assembly (e.g., a catheter), which typically passes through and/or remains in the introducer after the widest portion of the pump assembly has passed through the introducer. The space between the narrower portion of the pump assembly and the introducer may become filled with blood. Blood that clots inside an introducer presents a risk to the patient as forward movement of any device through the introducer may expel the blood clot into the patient's vasculature resulting in ischemia or stroke.

The existing solution to prevent clotting in the space between the introducer and the narrower portion of the pump assembly remaining in the introducer is to flush the introducer periodically or continuously. Adhering to a flushing schedule and making sure the continuous flush solution does not run out is a challenge for medical staff and a clot or clots may still form as a result. Also, if there is a kink in the introducer these flushing techniques may not be effective.

An additional structure that prevents blood from entering the space and a means to deposit the additional structure onto a narrower portion of the pump assembly after introduction of the pump into a patient is needed.

### BRIEF SUMMARY

The present disclosure describes systems, devices, and methods of loading an additional structure onto a catheter-based device after such a device has been percutaneously inserted into a patient.

In one aspect, the present disclosure describes a vascular access system comprising: a first sheath, a slitted sleeve, and a sealing portion of the slitted sleeve. The first sheath has a first sheath lumen extending between a proximal end and a distal end of the first sheath. The first sheath is configured to allow for passage of a first portion of a medical device. The medical device is configured to be inserted into a blood vessel. The first portion of the medical device has a first radial cross section, and a second portion of the medical device has a second radial cross section that is larger than the first radial cross section in at least one dimension. For simplicity, the first and second radial cross sections are referred to herein as the first and second widths, respectively, or the first and second diameters, respectively. The slitted sleeve comprises a tubular sleeve body having a longitudinal slit along its length. The slitted sleeve is adapted to receive the first portion of the medical device therein, which is loaded into the slitted sleeve through the longitudinal slit in the slitted sleeve. The tubular sleeve body has a first opening at a proximal end thereof and a second opening at a distal end thereof and a continuous lumen from the first opening to the second opening. The longitudinal slit closes, but does not seal, after the first portion of the medical device is inserted therein. The sealing portion of the slitted sleeve is configured to fill a gap and form a seal between an inner surface of the first sheath and a portion of the first portion of the medical device disposed in the first sheath when the first sheath is inserted in the blood vessel and the slitted sleeve is inserted into the first sheath lumen over the portion of the first portion of the medical device disposed in the first sheath, such that, in operation, blood from the blood vessel is substantially prevented from migrating past the seal.

In some embodiments, the sealing portion of the slitted sleeve of the vascular access system is a slitted sleeve tip at the distal open end of the tubular sleeve body. The slitted sleeve tip has an inner surface defining a slitted sleeve tip lumen that is in fluid communication with the slitted tubular sleeve body. In some embodiments of the vascular access system, an outer diameter of the proximal end of the slitted sleeve tip is larger than an outer diameter of the distal end of the slitted sleeve tip such that the slitted sleeve tip is tapered along its proximal-to-distal length. In some embodiments of the vascular access system, a diameter of the inner surface at the proximal end of the slitted sleeve tip is larger than a diameter of the inner surface of the slitted sleeve tip at the distal end. In some embodiments of the vascular access system, a diameter of the inner surface at the proximal end of the slitted sleeve tip is equal to a diameter of the inner surface at the distal end of the slitted sleeve tip. In some embodiments, the slitted sleeve tip of the vascular access system extends beyond the first sheath when the tubular sleeve body of the slitted sleeve is inserted into the first sheath lumen.

In some embodiments, the tubular sleeve body of the vascular access system comprises a first material and the slitted sleeve tip comprises a second material. In some embodiments, the first material of the vascular access system is substantially stiffer than the second material, and the second material is substantially more elastic than the first material. In some embodiments, the first material of the vascular access system comprises at least one of: high-density polyethylene (HDPE) material, a medium-density polyethylene (MDPE) material, a low-density polyethylene (LDPE) material, a polyether block amide (PEBA), a material with an elastic modulus of about 81-307 MPa, or a material with a yield strain of 20-30%. In some embodiments, the second material of the vascular access system comprises at least one of: ethylene-vinyl acetate (EVA), styrene-butadiene copolymer (SBC), synthetic rubber, an elastomer, an elastic material, a material with an elastic modulus of about 1.6 ksi, or a material with a yield strain in excess of 200%.

In some embodiments, the first portion of the medical device is a catheter that is coupled to a percutaneously insertable heart pump. In some embodiments, the slitted sleeve of the vascular access system is configured to slide distally along the catheter and be advanced into the blood vessel while assembled to the catheter. In some embodiments, the slitted sleeve of the vascular access system is configured to be inserted into the first sheath by moving the slitted sleeve and the first sheath axially relative to one another along a longitudinal axis of the slitted sleeve and the first sheath.

In some embodiments, both the slitted sleeve and the first sheath of the vascular access system are configured to be slidably coupled to the first portion of the medical device (e.g., catheter).

In some embodiments, the first sheath lumen of the vascular access system is configured to have a first inner diameter at rest, and to elastically expand from the first inner diameter to a second inner diameter during passage of the second portion of the medical device (i.e., the wider portion of the medical device) through the first sheath. The first sheath lumen is configured to contract from the second inner diameter to the first inner diameter after the passage of the second portion of the medical device therethrough and during passage of the first portion of the medical device (i.e., the portion of the medical device that is less wide than the second portion of the medical device), leaving a gap between an inner surface of the first sheath and an outer surface of the first portion of the medical device. In some embodiments, the first sheath of the vascular access system is configured to be expandable by blood pressure within the blood vessel so as to seal a space between an outer surface of the slitted sleeve and an arteriotomy in the blood vessel.

In some embodiments of the vascular access system, an outer diameter of the first sheath is dimensioned to be introduced through a percutaneous access site of about 20 Fr (6.67 mm) or less.

In some embodiments of the vascular access system, an outer surface of the slitted sleeve is coated with one of: an antithrombogenic coating, or a coating configured to reduce a likelihood of blood clot formation between the first sheath and the first portion of the medical device (e.g., a catheter) when they are inserted into the blood vessel. In some embodiments of the vascular access system, an outer surface of the first sheath is coated with one of: a hydrophilic coating, a hydrophobic coating, or a coating to reduce friction. In some embodiments of the vascular access system, an outer surface of the first sheath is coated with one of: an antimicrobial coating or a coating configured to reduce a likelihood of infection occurring in the vessel when the first sheath is inserted into the blood vessel.

In some embodiments, the vascular access system further comprises a slitted sleeve hub, wherein the slitted sleeve is received into a lumen of a first sheath hub.

In some embodiments, the slitted sleeve hub of the vascular access system comprises at least one of: a high-density polyethylene (HDPE) material, a medium-density polyethylene (MDPE) material, a low-density polyethylene (LDPE) material, polyether ether ketone (PEEK), or a polyether block amide (PEBA).

In some embodiments, the slitted sleeve hub and the first sheath hub of the vascular access system are configured to couple to each other via at least one of: a threaded connection, a press fit connection, or a cliplock connection. In some embodiments, the first sheath hub of the vascular access system includes a feature configured for suturing to a patient. In some embodiments, the feature of the vascular access system configured for suturing to the patient includes a pair of suture wings.

In some embodiments, the slitted sleeve hub comprises a hub slit, and the hub slit substantially aligns with the longitudinal slit in the slitted sleeve. In some embodiments, the slitted sleeve tip comprises a tip slit, and the tip slit substantially aligns with the longitudinal slit in the slitted sleeve.

In another aspect, the present disclosure describes a vascular access device accessory comprising a slitted sleeve, a closure, and a slitted sleeve hub, wherein the slitted sleeve is received into a lumen of the slitted sleeve hub. The slitted sleeve comprises a tubular sleeve body extending from the slitted sleeve hub and the slitted sleeve is adapted to receive a first portion of a medical device (e.g., a catheter), which is loaded into the slitted sleeve through a longitudinal slit in the slitted sleeve. The tubular sleeve body of the slitted sleeve defines a lumen that as a first opening at a proximal end thereof and a second opening at a distal end thereof. The proximal end of the slitted sleeve is received by the lumen of the slitted sleeve hub. The longitudinal slit is closable and the lumen of the slitted sleeve hub has a longitudinal opening through which the first portion of the medical device may be inserted. The closure is adapted to slide along the slitted sleeve and the first portion of the medical device and comprises an outer channel and an inner channel. The outer channel slidably engages the slitted sleeve and the inner channel slidably engages the first portion of the medical device. The outer channel has openings at a first end and a second end to receive the slitted sleeve. The inner channel has an opening to slidably receive the first portion of the medical device. The slitted sleeve hub comprises a receptacle for receiving and securing the closure therein.

In some embodiments, the vascular access device accessory further comprises the closure that does not have completely separate inner and outer channels. There is a division between the portion of the closure that receives the slitted sleeve and the portion of the closure through which the catheter passes. This division may be of many forms and, in one form, is in the form of radial walls that extend partially through the inner portion of the closure but do not close on themselves to define separate channels for the sleeve and catheter.

A method for inserting a vascular access device accessory into a sheath is contemplated. According to the method, a first sheath is inserted into the blood vessel through which a medical device is passed. The first sheath has a first sheath lumen extending between a proximal end and a distal end of the first sheath. The medical device has a first portion with a first radial cross section and a second portion with a second radial cross section that is larger than the first radial cross section. The cross section of the first sheath is larger than the first radial cross section.

According to the method, the second portion of the medical device with the second cross section is at least partially advanced through the first sheath. A portion of the first portion of the medical device is then inserted into a vascular access device accessory. The vascular access device accessory has a slitted sleeve having a tubular sleeve body having a longitudinal slit along its length and a first opening at a proximal end of the tubular sleeve body and a second opening at a distal end of the tubular sleeve body and a continuous lumen from the first opening to the second opening. The vascular access device accessory also has a slitted sleeve hub, wherein the proximal end of the slitted sleeve is received into a lumen of the slitted sleeve hub and wherein the slitted sleeve extends from the slitted sleeve hub, and a closure. The closure may have an outer channel and an inner channel, wherein the outer channel slidably engages the slitted sleeve. According to the method, a portion of the first portion of the medical device is inserted into the vascular device by: i) placing the first portion of the medical device adjacent to the slitted sleeve; ii) sliding the closure along at least a portion of the length of the sleeve thereby drawing a portion of the first portion of the medical device into the tubular sleeve body of the slitted sleeve through the longitudinal slit in the slitted sleeve; and iii) advancing the vascular access device accessory over the first portion of the medical device and into a proximal end of the first sheath lumen. In some embodiments, the method includes the steps of: i) sliding the closure across an entirety of the tubular sleeve body, thereby drawing the first portion of the medical device into the slitted sleeve along an entire length of the slitted sleeve; and ii) receiving the closure into the slitted sleeve hub to secure the closure. The method may further include advancing the slitted sleeve hub into a lumen of a first sheath hub. Advancing the slitted sleeve hub into the lumen of the first sheath hub may include coupling the slitted sleeve hub into the first sheath hub via at least one of a threaded connection, a press fit connection, a cliplock connection, locking pin, a clamp, a twist lock, a pop lock, or a snapping fit.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1(a) illustrates an exemplary embodiment of a vascular access device accessory of the present disclosure.

FIG. 1(b) illustrates the vascular access device accessory of FIG. 1(a) with a closure according to an embodiment of the present disclosure.

FIG. 1(c) illustrates an enlarged view of the distal end of the vascular access device accessory of FIG. 1(b).

FIG. 2 illustrates a vascular access device accessory partially coupled to a catheter via a closure according to an embodiment of the present disclosure.

FIG. 3 illustrates a vascular access system including a vascular access device accessory and an introducer according to an embodiment of the present disclosure.

FIG. 4(a) illustrates a vascular access system according to an embodiment of the present disclosure where the vascular access device accessory is docked within an introducer.

FIG. 4(b) illustrates a cross section of the vascular access system of FIG. 4(a).

FIG. 5 illustrates another embodiment of a vascular access device accessory of the present disclosure coupled to a catheter.

FIG. 6 illustrates a vascular access system wherein the embodiment of the vascular access device accessory of FIG. 5 is docked within an introducer sheath assembly.

FIG. 7 illustrates a closure device of the present disclosure according to one embodiment.

FIG. 8 illustrates a closure device of the present disclosure according to a second embodiment.

FIG. 9 illustrates a closure device of the present disclosure according to a third embodiment.

FIG. 10 illustrates stages of a method of coupling a vascular access device accessory to a catheter without a closure according to an embodiment of the present disclosure.

FIG. 11 illustrates an exemplary method of loading a vascular assembly onto a catheter with or without a closure according to an embodiment of the present disclosure.

FIGS. 12-14 illustrate alternative exemplary methods of the present disclosure of loading a vascular assembly onto a catheter with a closure.

### DETAILED DESCRIPTION

Aspects of the present disclosure are described in detail with reference to the drawing figures wherein like reference numerals identify similar or identical elements. It is to be understood that the disclosed aspects are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

As used herein, including in the claims, terms that denote shape, such as circle, circular or square, mean within reasonable manufacturing tolerances. Terms that denote relative position, such as coaxial or collinear, mean within reasonable manufacturing tolerances. Similarly, terms or phrases that denote dimensions, such as constant outside diameter along an object's length, mean within reasonable manufacturing tolerances.

As used herein, including in the claims, “tubular” does not necessarily mean having a circular cross section. A tubular item may, for example, have an oval, polygonal, irregular, or other shaped cross section.

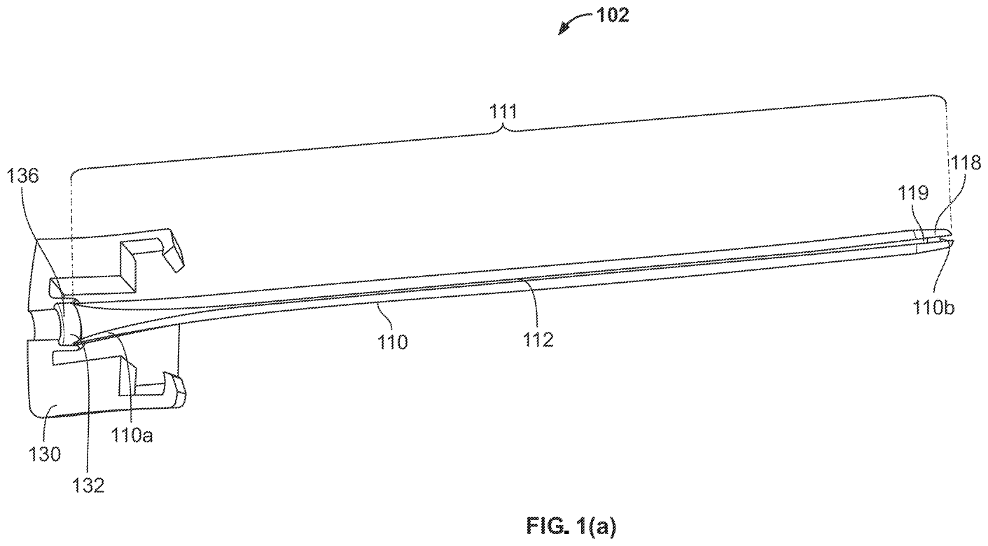

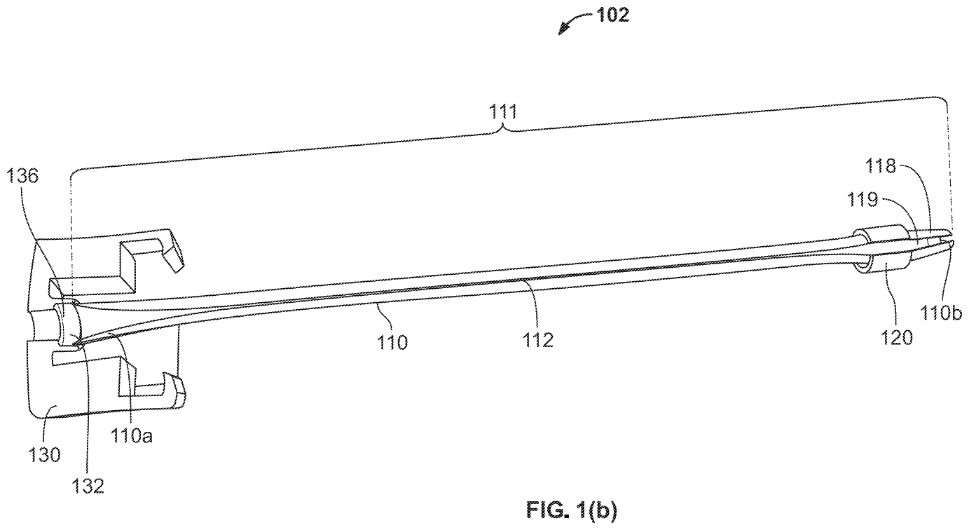

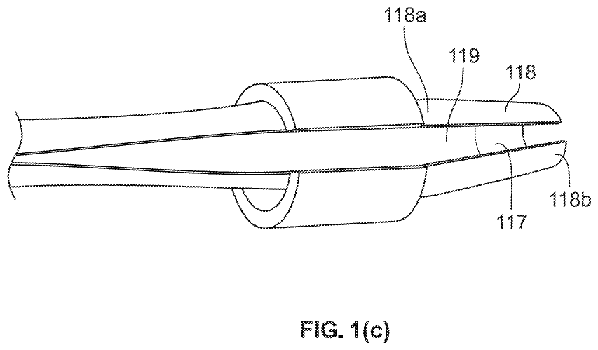

FIG. 1(a) illustrates a vascular access device accessory 102 of the present disclosure configured to be coupled to a medical device. In some embodiments the medical device is a percutaneously inserted intracardiac blood pump comprising a second portion that is a pump assembly and a first portion that is a catheter coupled thereto. The intracardiac blood pump is configured to be inserted into a blood vessel of a patient. In some embodiments, a width of one or more portions of the pump assembly is greater than a width of the catheter.

Vascular access device accessory 102 may include a slitted sleeve 110, a closure 120, and a slitted sleeve hub 130. The slitted sleeve 110 has longitudinal slit 112 and a tubular body 111 extending from a proximal end 110a of the slitted sleeve 110 to a distal end 110b of the slitted sleeve 110. Slitted sleeve 110 is constructed from a semi-stiff material that allows for some pliability. Such pliability enables longitudinal slit 112 to be closeable in some embodiments. For example, slitted sleeve 110 may be constructed of high-density polyethylene (HDPE) material, a medium-density polyethylene (MDPE) material, a low-density polyethylene (LDPE) material, a polyether block amide (PEBA), a material with an elastic modulus of about 81-307 MPa, or a material with a yield strain of 20-30%. The outer surface of slitted sleeve 110 may be coated. For example, slitted sleeve 110 may be coated with an antithrombogenic coating, or a coating configured to reduce a likelihood of blood clot formation between the first sheath and the catheter when they are inserted into the blood vessel.

The vascular access device accessory may further comprise a sealing portion of the slitted sleeve configured to fill a gap and form a seal between an inner surface of a first sheath and a portion of the first portion of the medical device disposed in the first sheath when the first sheath is inserted in a blood vessel and the slitted sleeve is inserted into a first sheath lumen over the portion of the first portion of the medical device disposed in the first sheath, such that, in operation, blood from the blood vessel is substantially prevented from migrating past the seal. In some embodiments, the sealing portion of the slitted sleeve is a slitted sleeve tip 118. In some embodiments, the slitted sleeve tip 118 is disposed on the distal end 110b of slitted sleeve 110. In other embodiments, the slitted sleeve tip 118 is disposed on another location of the slitted sleeve 110. In yet other embodiments, the sealing portion of the slitted sleeve is a separate component attached to the distal end 110b of slitted sleeve 110 or another location of the slitted sleeve 110. The slitted sleeve tip has an inner surface defining a slitted sleeve tip lumen 119 in fluid communication with the tubular body 111 of slitted sleeve 110. The proximal end 110a of the slitted sleeve 110 is received into a lumen 132 of a slitted sleeve hub 130. The lumen 132 of the slitted sleeve hub 130 contains a longitudinal opening 136 in line with longitudinal slit 112 of slitted sleeve 110. Longitudinal opening 136 of lumen 132 is in line with longitudinal slit 112 of slitted sleeve 110 such that a portion of a medical device may be received therein. FIG. 1(b) shows closure or zipper 120 of the present disclosure disposed on slitted sleeve 110 of the vascular access device accessory 102 of FIG. 1(a). FIG. 1(b) illustrates closure 120 disposed towards the distal end of slitted sleeve 110 of FIG. 1(a), however, closure 120 may be disposed at any point along the tubular body 111 of slitted sleeve 110.

FIG. 1(c) illustrates an enlarged view of the distal end 110b of the slitted sleeve 110 of a vascular access device accessory 102 of FIG. 1(b). Specifically, FIG. 1(c) shows a proximal end 118a of slitted sleeve tip 118 and a distal end 118b of slitted sleeve tip 118. Slitted sleeve tip 118 further contains a longitudinal opening 117 in line with longitudinal slit 112 and longitudinal opening 136 of lumen 132. In certain embodiments, the proximal end 118a and distal end 118b of slitted sleeve tip may have differing diameters. For example, the diameter of the proximal end 118a may be larger than the diameter of the distal end 118b such that slitted sleeve tip 118 is tapered. Alternatively, the diameter of the proximal end 118a may be the same as the diameter of the distal end 118b. Slitted sleeve tip 118 may be constructed from the same or similar material to slitted sleeve 110. Alternatively, slitted sleeve tip 118 may be constructed from a second material, the second material being substantially more elastic than the first material of the slitted sleeve 110. For example, the second material may be ethylene-vinyl acetate (EVA), styrene-butadiene copolymer (SBC), synthetic rubber, an elastomer, an elastic material, a material with an elastic modulus of about 1.6 ksi, or a material with a yield strain in excess of 200%.

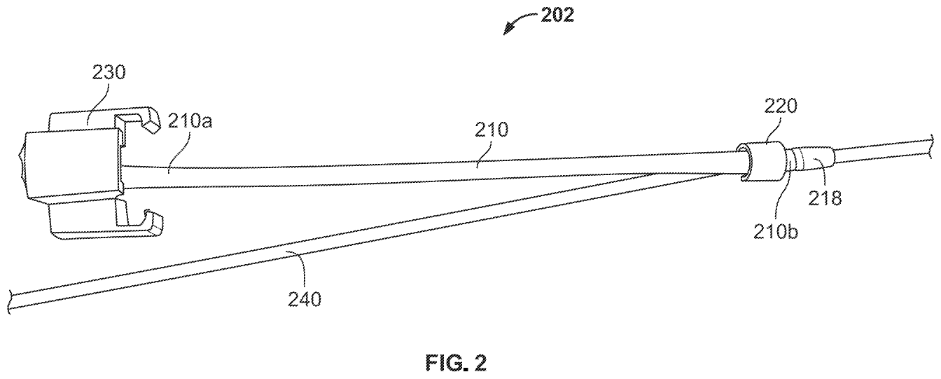

FIG. 2 shows an exemplary vascular access device accessory 202 of the present disclosure including a first portion of a medical device, e.g., catheter 240, at a first stage of connection. Catheter 240 is connected to the vascular access device accessory by closure 220 and slitted sleeve tip 218 at the distal end 210b of slitted sleeve 210. The proximal end 210a of slitted sleeve 210 along with slitted sleeve hub 230 remains detached from catheter 240.

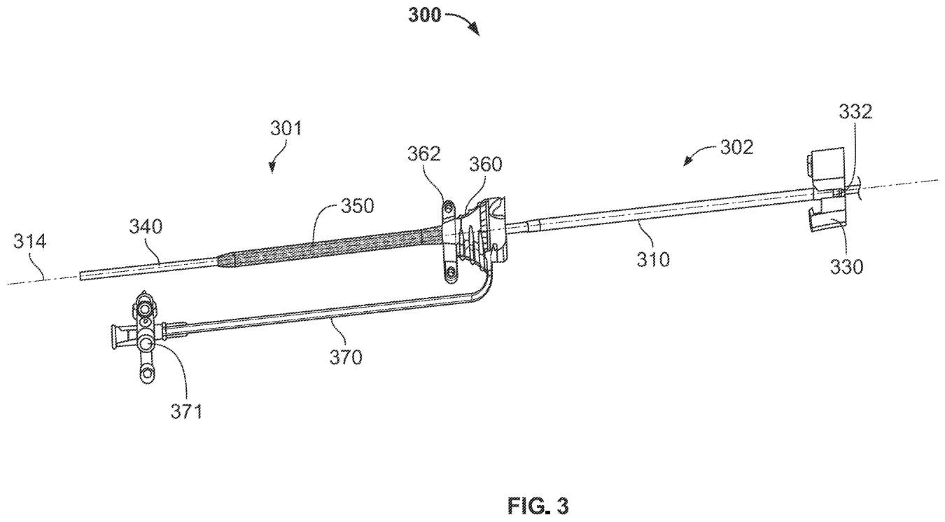

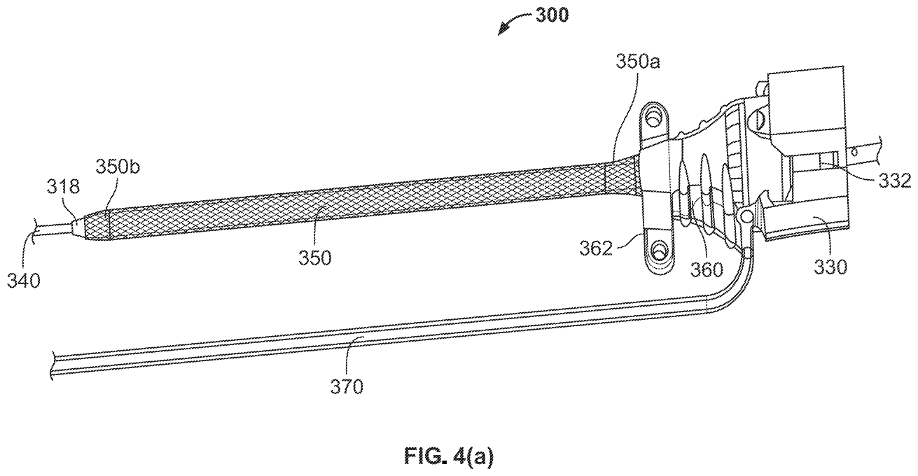

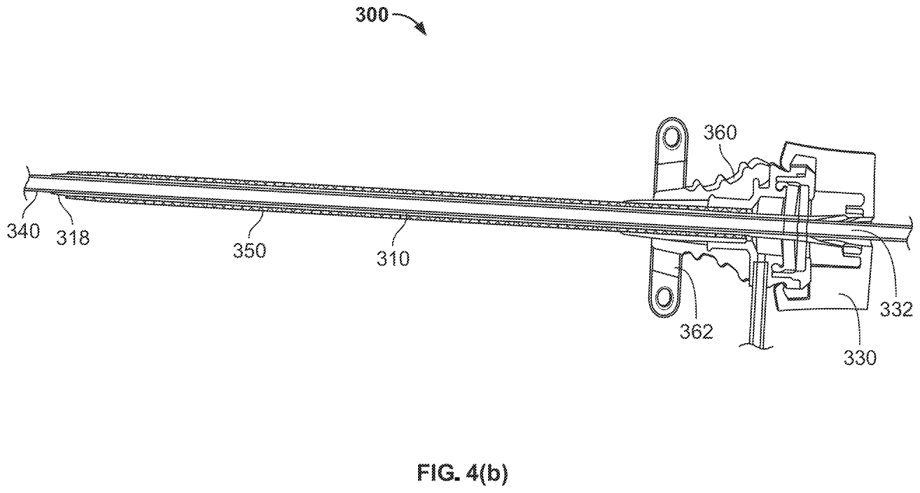

FIGS. 3, 4(a), and 4(b) illustrate embodiments of a vascular access system 300 of the present disclosure including a vascular access device accessory 302, an introducer 301, and a first portion of a medical device, e.g., catheter 340. Introducer 301 comprises a sheath 350 defined by a distal end 350b, a proximal end 350a, and a sheath lumen extending through sheath 350 between the proximal end 350a and distal end 350b. On the proximal end 350a of the sheath 350, the sheath hub 360 is attached to sheath 350. Sheath 350 may be coated with a hydrophilic coating, hydrophobic coating, or a coating to reduce friction. Sheath 350 may also be coated with an antimicrobial coating, or a coating that otherwise reduces a likelihood of infection occurring in the vessel when the sheath 350 is inserted into the blood vessel. Sheath 350 is configured to allow for passage of medical device therein. Both the slitted sleeve 310 of the vascular access device accessory 302 and the sheath of the introducer 301 are configured to slidably engage that medical device, including catheter 340.

In one embodiment, sheath 350 of introducer 301 has a fixed, predetermined diameter. The diameter is fixed along the entire length of the sheath 350. Where sheath 350 of introducer 301 is not radially expandable, the diameter must be large enough to accommodate the portion of a medical device with the largest width or diameter (e.g., pump assembly), even if other portions of the medical device (e.g., catheter) have significantly smaller widths/diameters.

In an alternative embodiment, sheath 350 of introducer 301 has a variable diameter. The diameter is variable along the entire length of the sheath 350. The sheath lumen of sheath 350 may have a first inner diameter at rest. The sheath lumen of sheath 350 may be configured to expand elastically to a second inner diameter when a second portion of the medical device passes though sheath lumen. The sheath lumen of sheath 350 may also be configured to contract from the second inner diameter to the first inner diameter when a first portion of the medical device passes through the sheath lumen. The first inner diameter is dimensioned to be greater than the diameter of the first portion of the medical device such that there is a gap between an inner surface of sheath 350 and an outer surface of the first potion of the medical device. For example, the second portion of the medical device may be a pump assembly of an intracardiac blood pump and a first portion of the medical device may be a catheter of the intracardiac blood pump. In another example, the second portion of the medical device may be a pump head and a pump body of an intracardiac blood pump and a first portion of the medical device could be a catheter of the intracardiac blood pump.

In one embodiment, the outer diameter of sheath 350 is no greater than about 20 Fr (6.67 mm). This allows sheath 350 to be introduced through a percutaneous access site of about 20 Fr (6.67 mm) or less.

Fluid may be introduced into the assembly via sidearm channel 370 (FIG. 3), and fluid flow into the device may be controlled by stopcock 371. A hemostatic valve (not shown) may also be included within hub 360, the hemostatic valve being configured to prevent blood from leaking outside of the patient during insertion and/or removal of a medical device (e.g., an intracardiac blood pump) or other components. In addition, in some implementations, the hub 360 may include a foam insert (not shown) placed proximal to the hemostatic valve that may be soaked with a lubricant such as silicone so that components will be lubricated as they are inserted through the foam and into the sheath body 350. Suture wings 362 (e.g., butterfly pads) are disposed on sheath hub 360. In one embodiment, suture wings 362 comprise a pair of suture wings, each disposed on either side of axis 314. Suture wings 362 are configured such that they may be used to suture vascular access system 300 to a patient.

FIG. 3 also illustrates vascular access device accessory 302 and introducer 301 of the present disclosure coupled to catheter 340. Catheter 340 is disposed within the slitted sleeve 310 and the lumen 332 of slitted sleeve hub 330 of the vascular access device accessory 302. In some embodiments of the present disclosure, slitted sleeve 310 is configured to be inserted into the sheath lumen of sheath 350. Slitted sleeve 310 may be moved axially along axis 314 and catheter 340 such that the slitted sleeve 310 passes through sheath hub 360 into the sheath lumen of sheath 350. Alternatively, the sheath 350 may be moved axially along axis 314 and catheter 340 until slitted sleeve 310 is inserted into the sheath lumen of sheath 350. Otherwise, slitted sleeve 310 and sheath 350 may be moved axially relative to one another along axis 314 and catheter 340. Slitted sleeve hub 330 may be configured to dock inside sheath hub 360 when slitted sleeve 310 is inserted into sheath 350.

In some embodiments, the slitted sleeve hub 330 may be configured to lock into the sheath hub 360 as shown in FIGS. 4(a) and 4(b). For example, slitted sleeve hub 330 may lock into sheath hub 360 using a threaded connection, a press fit connection, a cliplock connection, locking pin, a clamp, a twist lock, a pop lock, a snapping fit, etc.

FIG. 4(a) shows vascular access device accessory 300 when vascular access system 302 and introducer 301 are docked while coupled to catheter 340. When docked, slitted sleeve 310 is received into the lumen of sheath 350 and slitted sleeve hub 330 is received in sheath hub 360. In some embodiments, slitted sleeve tip 318 may extend beyond sheath 350 when slitted sleeve 310 is inserted into sheath 350.

FIG. 4(b) shows a cross sectional view of vascular access device accessory 300 when vascular access system 302 and introducer 301 are docked while coupled to catheter 340. Slitted sleeve 310 fills a gap between the outer diameter of catheter 340 and the inner diameter of sheath 350 at rest, substantially preventing blood from entering the gap. In some embodiments, when slitted sleeve tip 318 extends beyond sheath 350, the slitted sleeve tip 318 may be advanced into a blood vessel of a patient and act as a seal such that blood from the blood vessel is substantially prevented from migrating past the seal. Additionally, when slitted sleeve tip 318 is tapered, it may be advanced such that a portion of the slitted sleeve tip 318 with a diameter most similar to that of the diameter of the incision of the patient is disposed in the incision. A tapered slitted sleeve tip 318 is preferred as it acts as a more effective seal for incisions of varying size.

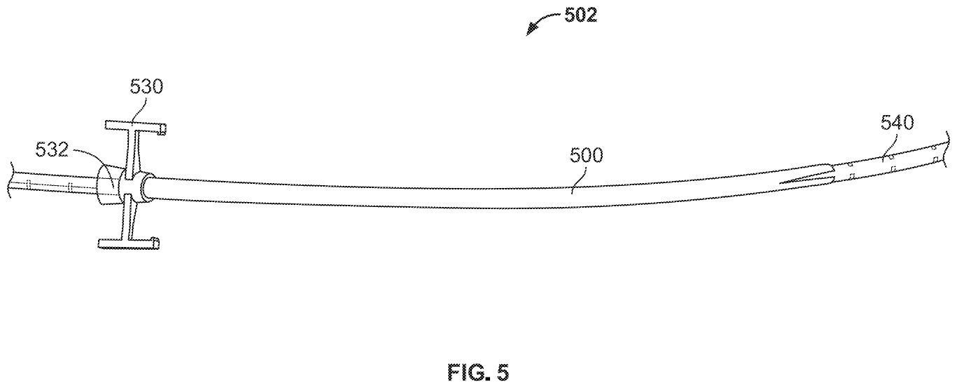

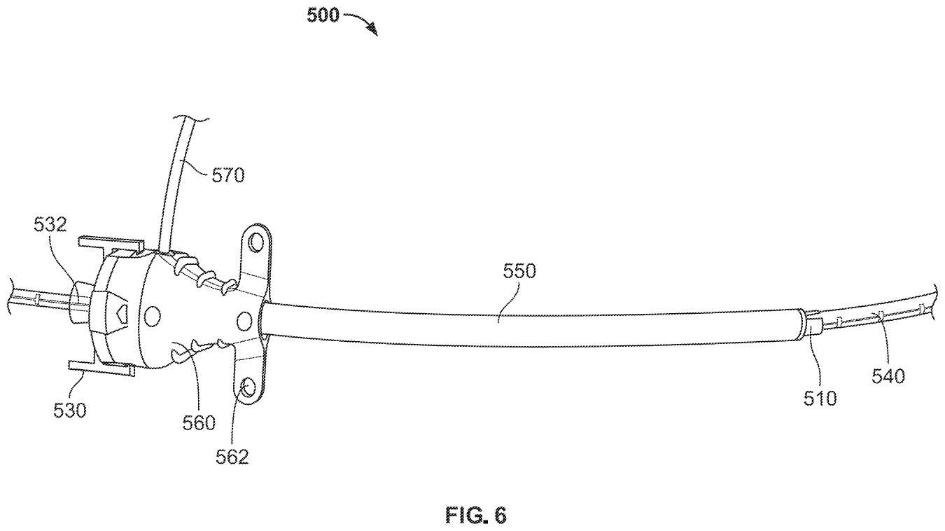

FIGS. 5 and 6 illustrate an alternative embodiment of a vascular access system 500 of the present disclosure. In FIG. 5, vascular access device accessory 502 is coupled to a portion of a medical device, e.g., catheter 540. Vascular access device accessory 502 including slitted sleeve 510, slitted sleeve hub 530 of the slitted sleeve hub 530. Catheter 540 is disposed within the slitted sleeve 510 and the lumen 532 of slitted sleeve hub 530 of the vascular access device accessory 502. FIG. 6 is the vascular access system 500 with the hub 530/sleeve 510 assembly of FIG. 5 inserted in the hub 560/sheath 550 assembly. FIG. 6 illustrates the vascular access system 500 where a vascular access device accessory is coupled to an introducer 550 sheath and catheter 540. Fluid may be introduced into the assembly via sidearm channel 570. In some embodiments where the vascular access system 500 is not sealed with the closure, fluid (e.g., blood) can also flow out, such as if the sidearm is used to help with distal perfusion.

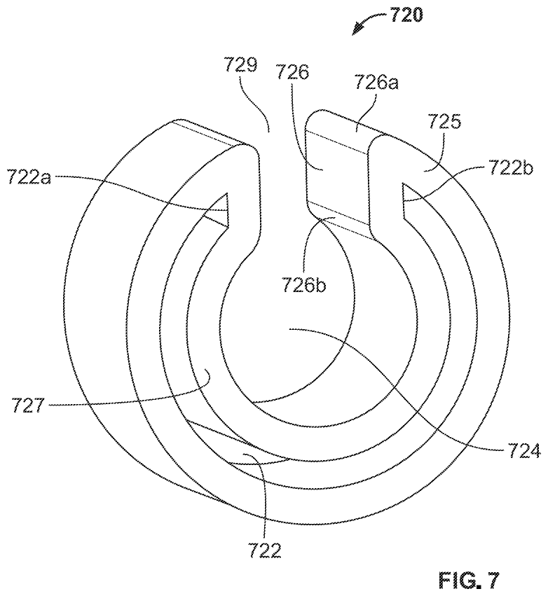

Closures described herein may have any suitable shape or geometry. For ease of description, the closures are illustrated as approximately annular in shape, but that is not a requirement. The closure may be annular, oval, polygonal, irregular or have another shape in cross section. The specific shape may be selected by the skilled person considering the form and function of the closure described herein, FIG. 7 shows an embodiment of closure 720 of the present disclosure. Closure 720 contains an outer channel 722 and an inner channel 724. Inner channel 724 is defined by an inner perimetral surface of inner wall 727. In one embodiment, closure 720 is circular and the outer channel 722 and inner channel 724 are defined by circumferential surfaces. Outer channel 722 is defined by an outer perimetral surface of inner wall 727, an inner perimetral surface of outer wall 725, a first radial surface 722a, and a second radial surface 722b, both the first radial surface 722a and the second radial surface 722b extending from the outer perimetral surface of inner wall 727 to the inner perimetral surface of outer wall 725. Closure 720 contains an opening 729 extending from the inner channel 724 through the outer perimetral surface of outer wall 725. The opening 729 is defined by the inner perimetral surface of inner wall 727, the outer perimetral surface of outer wall 725, and radial walls. The radial walls extend from the inner perimetral surface of inner wall 727 to the outer perimetral surface of the outer wall 725. The radial walls 726 have a first edge and a second edge at the inner perimetral surface of inner wall 727 and the outer perimetral surface of the outer wall 725, respectively. For example, radial wall 726 has a first rounded edge 726a at the outer perimetral surface of the wall 725 and a second rounded edge 726b at the inner perimetral surface of the wall 727. In some embodiments, the perimetral surfaces are circular in shape.

Closure 720 is configured to slidably engage the tubular body of the slitted sleeve and the catheter of a medical device. Closure 720 has openings on both faces such that the slitted sleeve may be received into outer channel 722. Following the insertion of the slitted sleeve into the outer channel 722, a catheter may be received into inner channel 724 through opening 729 and rest on the inner perimetral surface of inner wall 727. Closure 720 is further configured to be advanced along the length on the tubular body of slitted sleeve and the catheter such that the slitted sleeve is deposited on the catheter as seen in, for example, FIG. 3. Additionally, closure 720 is configured to be received and docked in the slitted sleeve hub after closure 720 has been advanced along the length of the tubular sleeve body of the slitted sleeve.

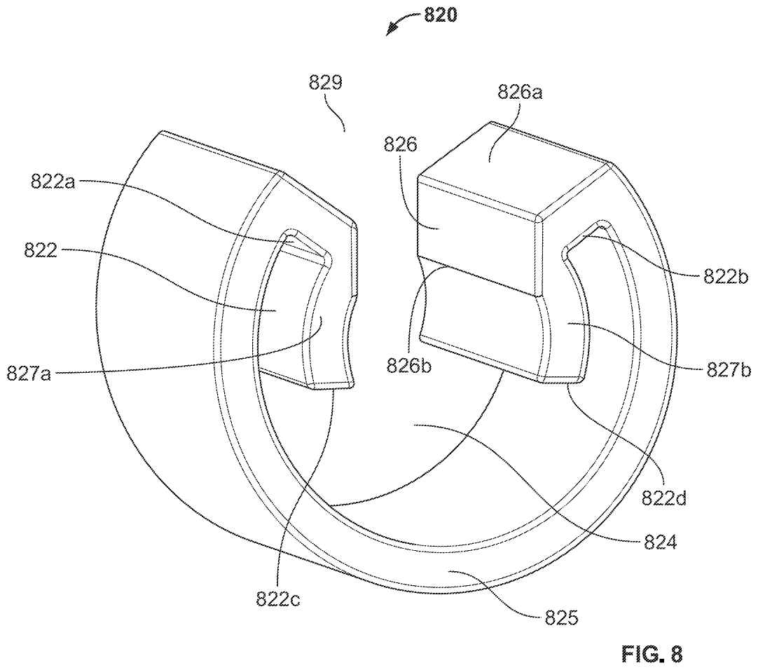

FIG. 8 illustrates a second embodiment of the closure 820 of the present disclosure. Closure 820 contains an outer channel 822 and an inner channel 824. Inner channel 824 is defined by an inner perimetral surface of inner wall 827a, 827b. Outer channel 822 is defined by the interior surface of inner walls 827a, 827b, an inner perimetral surface of outer wall 825, a first radial surface 822a, and a second radial surface 822b, both the first radial surface 822a and the second radial surface 822b extending from the outer perimetral surface of inner wall 827 to the inner perimetral surface of outer wall 825. The first inner wall 827a and the second inner wall 827b may be the same size and the same shape. Each inner wall 827a, 827b may extend from a radial wall, such as radial wall 826, to a third radial surface 822c and a fourth radial surface 822d, respectively. The third radial surface 822c and the fourth radial surface 822d extend from the outer perimetral surface of inner walls 827a, 827b to inner perimetral surface of inner walls 827a, 827b. Closure 820 contains a radial opening 829 extending from the inner channel 824 through the outer perimetral surface of outer wall 825. The radial opening 829 defined by the inner perimetral surface of inner walls 827a, 827b, the outer perimetral surface of outer wall 825, radial walls, and flat edge surfaces. The radial walls extend from the inner perimetral surface of inner walls 827a, 827b to the outer perimetral surface of the inner walls 827a, 827b. The radial walls have a first edge and a second edge. For example, radial wall 826 has a first flat edge 826a extending from the outer perimetral surface of outer wall 825 to the outer perimetral surface of inner wall 827 and a second edge 826b at the inner perimetral surface of the wall 827. In some embodiments, the perimetral surfaces are circular in shape.

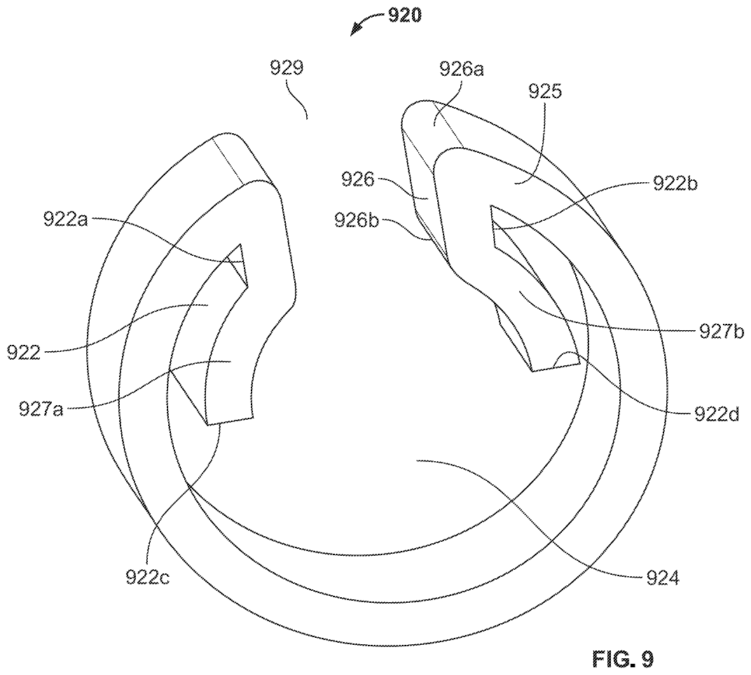

FIG. 9 depicts a third embodiment of the closure 920 of the present disclosure. Closure 920 contains an outer channel 922 and an inner channel 924. Inner channel 924 is defined by an inner perimetral surface of inner wall 927. Outer channel 922 is defined by an outer perimetral surface of inner wall 927a, 927b, an inner perimetral surface of outer wall 925, a first radial surface 922a, and a second radial surface 922b, both the first radial surface 922a and the second radial surface 922b extending from the outer perimetral surface of inner wall 927 to the inner perimetral surface of outer wall 925. The first inner wall 927a and the second inner wall 927b may be the same size and the same shape. Each portion of inner walls 927a, 927b may extend from a radial wall, such as radial wall 926, to a third radial surface 922c and a fourth radial surface 922d, respectively. The third radial surface 922c and fourth radial surface 922d extend from the outer perimetral surface of inner walls 927a, 927b to inner perimetral surface of inner walls 927a, 927b. Closure 920 contains a radial opening 929 extending from the inner channel 924 through the outer perimetral surface of outer wall 925. The radial opening 929 is defined by the inner perimetral surface of inner wall 927, the outer perimetral surface of outer wall 925, and radial walls. The radial walls have a first edge and a second edge at the inner perimetral surface of inner walls 927a, 927b and the outer perimetral surface of the wall 925, respectively. For example, radial wall 926 has a first rounded edge 926a at the outer perimetral surface of the wall 925 and a second rounded edge 926b at the inner perimetral surface of the wall 927. In some embodiments, the perimetral surfaces are circular in shape.

The operation of the closure of the present disclosure illustrated in FIGS. 8 and 9 is similar to the operation of the closure of FIG. 7, but the closures of FIGS. 8 and 9 do not have completely separate channels through which sleeve slides and the catheter is drawn into the sleeve. For convenience, the operation of the closures in FIGS. 8 and 9 are described collectively, using the corresponding reference numerals from FIGS. 8 and 9, respectively. Closure 820, 920 has openings on both faces wherein the slitted sleeve may be received into outer channel 822, 922. Alternatively, the slitted sleeve may be received into outer channel 822, 922 through opening 829, 929 and then manipulated for the slitted sleeve to extend into channels 822, 922. A catheter of a medical device may be received into inner channel 824, 924 through opening 829, 929 and rest on the inner surface of the slitted sleeve in addition to the inner perimetral surfaces of inner walls 827a, 927a, 827b, 927b. When positioned in the closure 820, 920, the slitted sleeve is slidably engaged therewith and with the catheter received therein. Closure 820, 920 is further configured to be advanced along the length of the tubular body of the slitted sleeve and the catheter such that the slitted sleeve receives the catheter, as seen in, for example, FIG. 3. Additionally, closure 820, 920 is configured to be received and docked in the slitted sleeve hub after closure 820, 920 has been advanced along the length of the tubular sleeve body of the slitted sleeve.

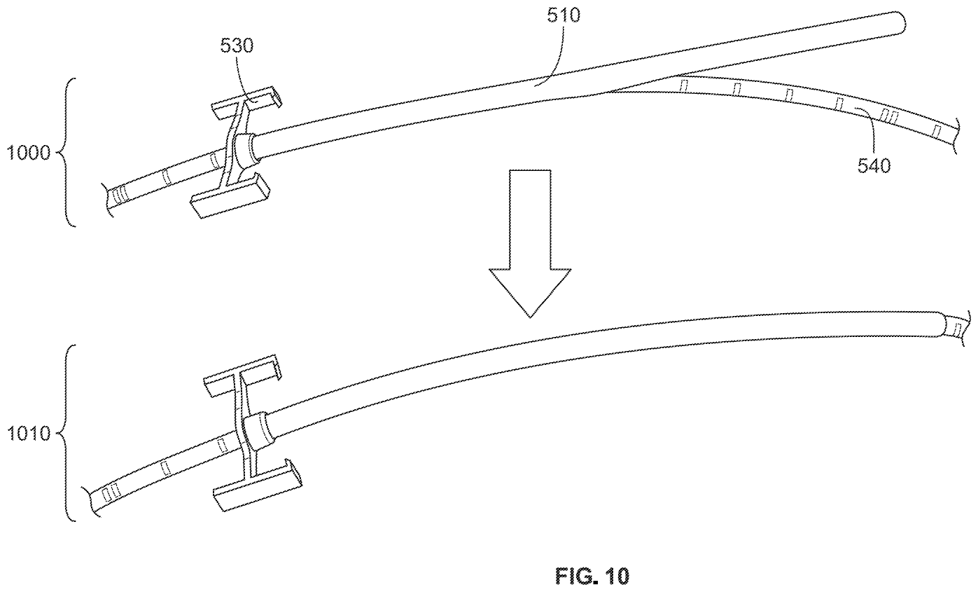

FIG. 10 illustrates stages 1000 and 1010 of a method of the present disclosure of coupling a vascular access device accessory 502 to a first portion of a medical device, e.g., catheter 540. At stage 1000, slitted sleeve hub 530 and a portion of slitted sleeve 510 are disposed on catheter 540. Between stages 1000 and 1001, a user presses slitted sleeve 510 onto catheter 540. As a result, at stage 1010, the entirety of slitted sleeve 510 and slitted sleeve hub 530 are coupled to catheter 540.

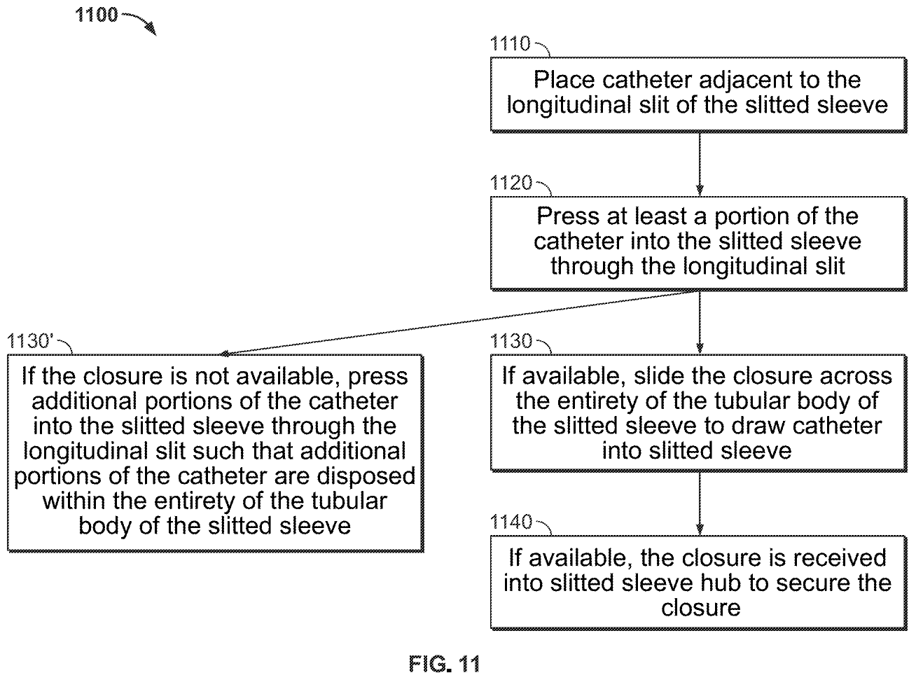

FIG. 11 describes an exemplary method 1100 of the present disclosure of inserting a first portion of a medical device, such as the catheter portion of a medical device, into the vascular access assembly. Step 1110 describes placing a catheter adjacent to the longitudinal slit of the slitted sleeve of the assembly. Step 1120 describes pressing at least a of portion of the catheter into the slitted sleeve through the longitudinal slit of the vascular access device accessory. If a closure is available, in step 1130, the closure is slid across the entirety of the tubular body of the slitted sleeve to draw the catheter into the slitted sleeve. Following step 1130, in step 1140, the closure is received into the slitted sleeve hub to secure the annular sleeve in place. Alternatively, if a closure is not available, as in step 1130′, additional portions of the catheter are manually pressed into the longitudinal slit such that the additional portions of the catheter are disposed within the entire length of the tubular body of the slitted sleeve.

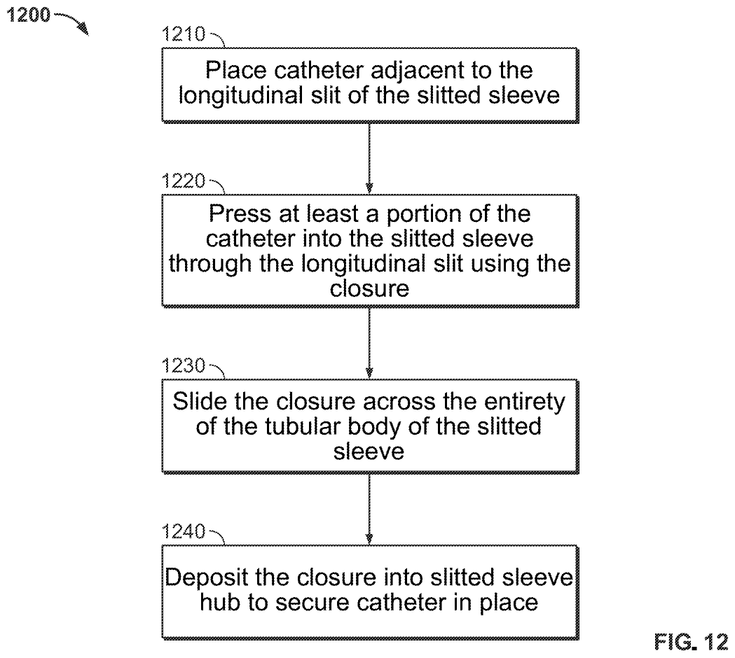

An alternative method of insertion 1200 of the present disclosure is shown in FIG. 12. In step 1210, the catheter is placed adjacent to the longitudinal slit of the slitted sleeve of a vascular access device accessory. Then, in step 1220, at least a portion of the catheter is pressed into the slitted sleeve through the longitudinal slit using the closure. In step 1230, the closure is slid across the entirety of the tubular body of the slitted sleeve, thereby drawing the catheter into the slitted sleeve along its entire length. In step 1240, the closure is received into the slitted sleeve hub to secure the catheter in place.

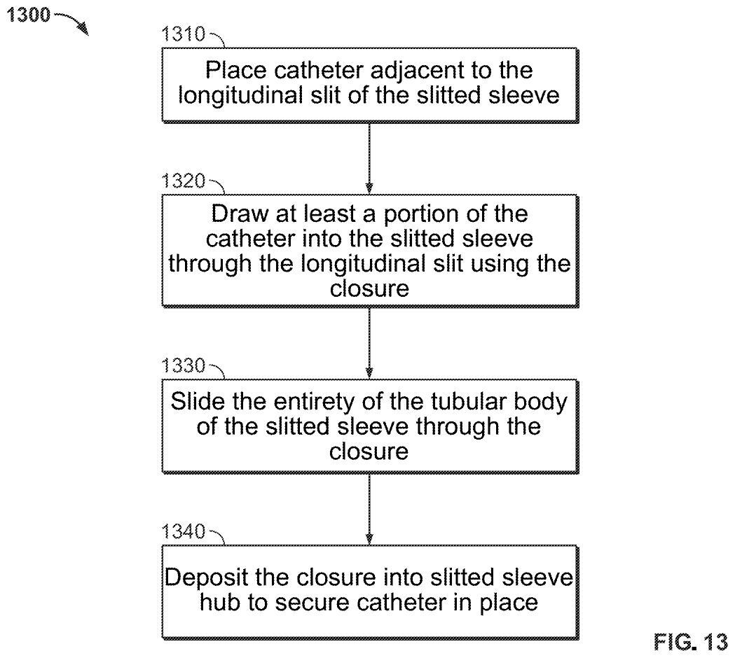

Another alternative method of insertion 1300 of the present disclosure is illustrated in FIG. 13. In step 1310, the catheter is placed adjacent to the longitudinal slit of the slitted sleeve of a vascular access device accessory. Then, in step 1320, at least a portion of the catheter is pressed into the slitted sleeve through the longitudinal slit using the closure. In step 1330, the tubular body of the slitted sleeve is slid through the closure to draw the catheter into the entire length of the slitted sleeve. In step 1340, the closure is received into the slitted sleeve hub to secure the closure in the slitted sleeve hub.

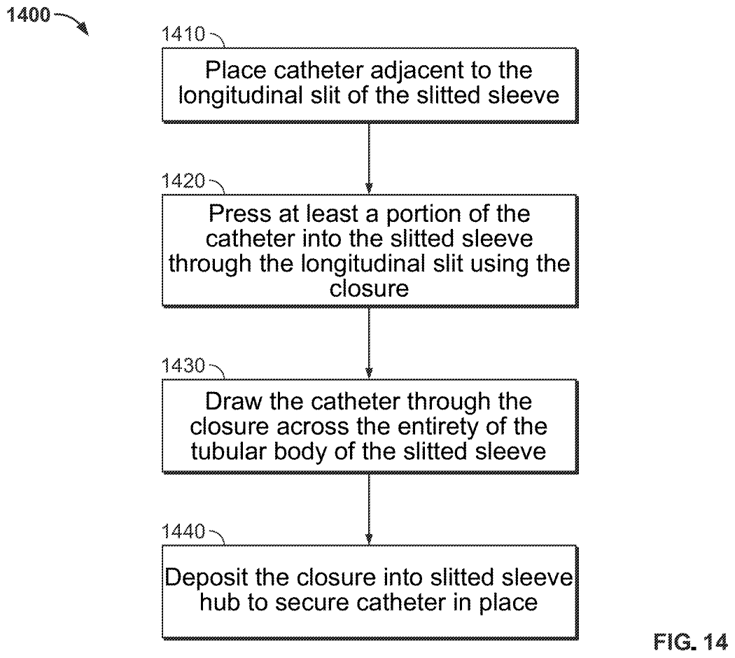

An additional alternative method of insertion 1400 of the present disclosure is described in FIG. 14. In step 1410, the catheter is placed adjacent to the longitudinal slit of the slitted sleeve of a vascular access device accessory. Then, in step 1420, at least a portion of the catheter is pressed into the slitted sleeve through the longitudinal slit using the closure. In step 1430, the catheter is slid through the closure across the entirety of the tubular body of the slitted sleeve. In step 1440, the closure is received into the slitted sleeve hub to secure the closure in the slitted sleeve hub.

In one aspect, described is a vascular access system comprising: i) a first sheath having a first sheath lumen extending between a proximal end and a distal end of the first sheath, the first sheath configured to allow for passage of a first portion of a medical device, wherein the medical device is configured to be inserted into a blood vessel, the first portion of the medical device having a first radial cross section, and a second portion of the medical device having a second radial cross section that is larger than the first radial cross section; ii) a slitted sleeve, wherein the slitted sleeve comprises a tubular sleeve body having a longitudinal slit along its length, wherein the slitted sleeve is adapted to receive the first portion of the medical device therein, which is loaded into the slitted sleeve through the longitudinal slit in the slitted sleeve, the tubular sleeve body having a first opening at a proximal end thereof and a second opening at a distal end thereof and a continuous lumen from the first opening to the second opening, and wherein the longitudinal slit closes, but does not seal, after the first portion of the medical device is inserted therein; and iii) a sealing portion of the slitted sleeve configured to fill a gap and form a seal between an inner surface of the first sheath and a portion of the first portion of the medical device disposed in the first sheath when the first sheath is inserted in the blood vessel and the slitted sleeve is inserted into the first sheath lumen over the portion of the first portion of the medical device disposed in the first sheath, such that, in operation, blood from the blood vessel is substantially prevented from migrating past the seal.

In one aspect, the sealing portion of the slitted sleeve is a slitted sleeve tip at the distal open end of the tubular sleeve body, the slitted sleeve tip having an inner surface defining a slitted sleeve tip lumen that is in fluid communication with the slitted tubular sleeve body.

In any of the above aspects, an outer diameter of the proximal end of the slitted sleeve tip is larger than an outer diameter of the distal end of the slitted sleeve tip such that the slitted sleeve tip is tapered along its proximal-to-distal length.

In any of the above aspects, a diameter of the inner surface at the proximal end of the slitted sleeve tip is larger than a diameter of the inner surface of the slitted sleeve tip at the distal end.

In any of the above aspects, a diameter of the inner surface at the proximal end of the slitted sleeve tip is equal to a diameter of the inner surface at the distal end of the slitted sleeve tip.

In any of the above aspects, the slitted sleeve tip extends beyond the first sheath when the tubular sleeve body of the slitted sleeve is inserted into the first sheath lumen.

In any of the above aspects, the tubular sleeve body comprises a first material and the slitted sleeve tip comprises a second material.

In any of the above aspects, the first material is substantially stiffer than the second material, and the second material is substantially more elastic than the first material.

In any of the above aspects, the first material comprises at least one of: high-density polyethylene (HDPE) material, a medium-density polyethylene (MDPE) material, a low-density polyethylene (LDPE) material, a polyether block amide (PEBA), a material with an elastic modulus of about 81-307 MPa, or a material with a yield strain of 20-30%.

In any of the above aspects, the second material comprises at least one of: ethylene-vinyl acetate (EVA), styrene-butadiene copolymer (SBC), synthetic rubber, an elastomer, an elastic material, a material with an elastic modulus of about 1.6 ksi, or a material with a yield strain in excess of 200%.

In any of the above aspects, the first portion of the medical device is a catheter coupled to a percutaneous heart pump.

In any of the above aspects, the slitted sleeve is configured to slide distally along the catheter and be advanced into the blood vessel while assembled to the catheter.

In any of the above aspects, the slitted sleeve is configured to be inserted into the first sheath by moving the slitted sleeve and the first sheath axially relative to one another along a longitudinal axis of the slitted sleeve and the first sheath.

In any of the above aspects, an outer surface of the slitted sleeve is coated with at least one of: an antithrombogenic coating, or a coating configured to reduce a likelihood of blood clot formation between the first sheath and the first portion of the medical device when they are inserted into the blood vessel.

In any of the above aspects, an outer surface of the first sheath is coated with one of: a hydrophilic coating, a hydrophobic coating, or a coating to reduce friction.

In any of the above aspects, an outer surface of the first sheath is coated with one of: an antimicrobial coating, or a coating configured to reduce a likelihood of infection occurring in the vessel when the first sheath is inserted into the blood vessel.

In any of the above aspects, the vascular access system further comprises a slitted sleeve hub, wherein the slitted sleeve is received into a lumen of a first sheath hub.

In any of the above aspects, the slitted sleeve hub comprises at least one of: a high-density polyethylene (HDPE) material, a medium-density polyethylene (MDPE) material, a low-density polyethylene (LDPE) material, polyether ether ketone (PEEK), or a polyether block amide (PEBA).

In any of the above aspects, the slitted sleeve hub and the first sheath hub are configured to couple to each other via at least one of a threaded connection, a press fit connection, or a cliplock connection.

In any of the above aspects, the slitted sleeve hub comprises a hub slit, and wherein the hub slit substantially aligns with the longitudinal slit in the slitted sleeve.

In any of the above aspects, the slitted sleeve tip comprises a tip slit, and wherein the tip slit substantially aligns with the longitudinal slit in the slitted sleeve.

In another aspect, described is a vascular access device accessory comprising: i) a slitted sleeve; ii) a slitted sleeve hub, wherein the slitted sleeve is received into a lumen of the slitted sleeve hub; and iii) a closure. The slitted sleeve comprises a tubular sleeve body having a longitudinal slit along its length, wherein the slitted sleeve extends from the slitted sleeve hub, wherein the slitted sleeve is adapted to receive a first portion of a medical device therein which is loaded into the slitted sleeve through the longitudinal slit in the slitted sleeve, the tubular sleeve body having a first opening at a proximal end thereof and a second opening at a distal end thereof and a continuous lumen from the first opening to the second opening, the proximal end of the slitted sleeve being received by the lumen of the slitted sleeve hub. The longitudinal slit closes, but does not seal, after the first portion of the medical device is inserted therein. The closure is adapted to slide along the slitted sleeve and the first portion of the medical device and comprises: an outer channel and an inner channel, wherein the outer channel slidably engages the slitted sleeve and the inner channel slidably engages the first portion of the medical device such that sliding the closure along a length of the slitted sleeve draws the first portion of the medical device into the slitted sleeve. The slitted sleeve hub comprises a receptacle for receiving and securing the closure therein.

In another aspect, the outer channel is at least partially defined by an inner side of an outer wall and an outer side of an inner wall of the closure, and wherein the inner channel communicates with a radial opening in the closure through which the first portion of the medical device is drawn, wherein the inner channel is at least partially defined by an inner side of the inner wall of the closure.

In any of the above another aspects, the inner wall of the closure has two portions, each of which extends from a side of the radial opening, each portion extending between the first channel and the second channel, wherein the two portions are not connected.

In any of the above another aspects, the radial opening has a first surface extending from an outer surface of the outer wall to the inner surface of the inner wall and wherein each inner wall has an inner surface that forms an annular surface in the annular opening.

In any of the above another aspects, the radial opening is formed in the outer wall and extends therethrough and wherein each of the two portions of the inner wall extend from opposite sides of the radial opening and between the first channel and the second channel, wherein the two portions are not connected.

In any of the above another aspects, each inner wall has a first inner surface that forms an annular surface in the annular opening and a second inner surface that partially separates the first channel and the second channel.

In any of the above another aspects, the second inner surface of each of the two portions of the inner wall are curvilinear.

In any of the above another aspects, the vascular access device accessory further comprises comprising a sealing portion of the slitted sleeve configured to fill a gap and form a seal between an inner surface of a first sheath and a portion of the first portion of the medical device disposed in the first sheath when the first sheath is inserted in a blood vessel and the slitted sleeve is inserted into a first sheath lumen over the portion of the first portion of the medical device disposed in the first sheath, such that, in operation, blood from the blood vessel is substantially prevented from migrating past the seal.

In any of the above another aspects, the sealing portion of the slitted sleeve is a slitted sleeve tip at the distal open end of the tubular sleeve body, the slitted sleeve tip having an inner surface defining a slitted sleeve tip lumen that is in fluid communication with the slitted tubular sleeve body.

In yet another aspect, described is a method for inserting a vascular access device accessory into a first sheath, the method comprising the steps of: i) inserting a first sheath into the blood vessel through which a medical device is passed, wherein the first sheath has a first sheath lumen extending between a proximal end and a distal end of the first sheath and wherein the medical device has a first portion with a first radial cross section and a second portion with a second radial cross section that is larger than the first radial cross section and wherein the cross section of the first sheath is larger than the first radial cross section; ii) advancing the second portion of the medical device with the second cross section at least partially through the first sheath; iii) inserting a portion of the first portion of the medical device into a vascular access device accessory; and iv) advancing the vascular access device accessory over the first portion of the medical device and into a proximal end of the first sheath lumen. The vascular access device accessory may comprise: a) a slitted sleeve comprising a tubular sleeve body having a longitudinal slit along its length and a first opening at a proximal end of the tubular sleeve body and a second opening at a distal end of the tubular sleeve body and a continuous lumen from the first opening to the second opening; b) a slitted sleeve hub, wherein the proximal end of the slitted sleeve is received into a lumen of the slitted sleeve hub and wherein the slitted sleeve extends from the slitted sleeve hub; and c) a closure, comprising an outer channel and an inner channel, wherein the outer channel slidably engages the slitted sleeve; wherein a portion of the first portion of the medical device is inserted into the vascular device accessory by: 1) placing the first portion of the medical device adjacent to the longitudinal slit of the slitted sleeve; 2) placing a portion of the first portion of the medical device into the inner channel of the closure; 3) sliding the closure along at least a portion of the length of the slitted sleeve thereby drawing the first portion of the medical device into the tubular sleeve body of the slitted sleeve through the longitudinal slit in the slitted sleeve.

In yet another aspect, inserting a portion of the first portion of the medical device into the vascular access device accessory further comprises receiving the closure into the slitted sleeve hub to secure the closure.

In any of the above yet another aspects, the method further comprises advancing the slitted sleeve hub into a lumen of a first sheath hub.

In any of the above yet another aspects, advancing the slitted sleeve hub into the lumen of the first sheath hub comprises coupling the slitted sleeve hub into the first sheath hub via at least one of a threaded connection, a press fit connection, a cliplock connection, locking pin, a clamp, a twist lock, a pop lock, or a snapping fit.

From the foregoing and with reference to the various figure drawings, those skilled in the art will appreciate that certain modifications may also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A vascular access system comprising:
a first sheath having a first sheath lumen extending between a proximal end and a distal end of the first sheath, the first sheath configured to allow for passage of a first portion of a medical device, wherein the medical device is configured to be inserted into a blood vessel, the first portion of the medical device having a first radial cross section, and a second portion of the medical device having a second radial cross section that is larger than the first radial cross section;
a slitted sleeve, wherein the slitted sleeve comprises a tubular sleeve body having a longitudinal slit along its length, wherein the slitted sleeve is adapted to receive the first portion of the medical device therein, which is loaded into the slitted sleeve through the longitudinal slit in the slitted sleeve, the tubular sleeve body having a first opening at a proximal end thereof and a second opening at a distal end thereof and a continuous lumen from the first opening to the second opening, and wherein the longitudinal slit closes, but does not seal, after the first portion of the medical device is inserted therein; and
a sealing portion of the slitted sleeve configured to fill a gap and form a seal between an inner surface of the first sheath and a portion of the first portion of the medical device disposed in the first sheath when the first sheath is inserted in the blood vessel and the slitted sleeve is inserted into the first sheath lumen over the portion of the first portion of the medical device disposed in the first sheath, such that, in operation, blood from the blood vessel is substantially prevented from migrating past the seal.

2. The vascular access system of claim 1, wherein the sealing portion of the slitted sleeve is a slitted sleeve tip at the distal open end of the tubular sleeve body, the slitted sleeve tip having an inner surface defining a slitted sleeve tip lumen that is in fluid communication with the slitted tubular sleeve body.

3. The vascular access system of claim 2, wherein an outer diameter of the proximal end of the slitted sleeve tip is larger than an outer diameter of the distal end of the slitted sleeve tip such that the slitted sleeve tip is tapered along its proximal-to-distal length.

4. The vascular access system of claim 2, wherein a diameter of the inner surface at the proximal end of the slitted sleeve tip is larger than a diameter of the inner surface of the slitted sleeve tip at the distal end.

5. The vascular access system of claim 2, wherein a diameter of the inner surface at the proximal end of the slitted sleeve tip is equal to a diameter of the inner surface at the distal end of the slitted sleeve tip.

6. The vascular access system of claim 2, wherein the slitted sleeve tip extends beyond the first sheath when the tubular sleeve body of the slitted sleeve is inserted into the first sheath lumen.

7. The vascular access system of claim 2, wherein the tubular sleeve body comprises a first material and the slitted sleeve tip comprises a second material.

8. The vascular access system of claim 7, wherein the first material is substantially stiffer than the second material, and the second material is substantially more elastic than the first material.

9. The vascular access system of claim 7, wherein the first material comprises at least one of: high-density polyethylene (HDPE) material, a medium-density polyethylene (MDPE) material, a low-density polyethylene (LDPE) material, a polyether block amide (PEBA), a material with an elastic modulus of about 81-307 MPa, or a material with a yield strain of 20-30%.

10. The vascular access system of claim 7, wherein the second material comprises at least one of: ethylene-vinyl acetate (EVA), styrene-butadiene copolymer (SBC), synthetic rubber, an elastomer, an elastic material, a material with an elastic modulus of about 1.6 ksi, or a material with a yield strain in excess of 200%.

11. The vascular access system of claim 1, wherein the first portion of the medical device is a catheter coupled to a percutaneous heart pump.

12. The vascular access system of claim 11, wherein the slitted sleeve is configured to slide distally along the catheter and be advanced into the blood vessel while assembled to the catheter.

13. The vascular access system of claim 1, wherein the slitted sleeve is configured to be inserted into the first sheath by moving the slitted sleeve and the first sheath axially relative to one another along a longitudinal axis of the slitted sleeve and the first sheath.

14. The vascular access system of claim 1, wherein an outer surface of the slitted sleeve is coated with at least one of: an antithrombogenic coating, or a coating configured to reduce a likelihood of blood clot formation between the first sheath and the first portion of the medical device when they are inserted into the blood vessel.

15. The vascular access system of claim 1, wherein an outer surface of the first sheath is coated with one of: a hydrophilic coating, a hydrophobic coating, or a coating to reduce friction.

16. The vascular access system of claim 1, wherein an outer surface of the first sheath is coated with one of: an antimicrobial coating, or a coating configured to reduce a likelihood of infection occurring in the vessel when the first sheath is inserted into the blood vessel.

17. The vascular access system of claim 1, further comprising a slitted sleeve hub, wherein the slitted sleeve is received into a lumen of a first sheath hub.

18. The vascular access system of claim 17, wherein the slitted sleeve hub comprises at least one of: a high-density polyethylene (HDPE) material, a medium-density polyethylene (MDPE) material, a low-density polyethylene (LDPE) material, polyether ether ketone (PEEK), or a polyether block amide (PEBA).

19. The vascular access system of claim 17, wherein the slitted sleeve hub and the first sheath hub are configured to couple to each other via at least one of a threaded connection, a press fit connection, or a cliplock connection.

20. The vascular access system of claim 17, wherein the slitted sleeve hub comprises a hub slit, and wherein the hub slit substantially aligns with the longitudinal slit in the slitted sleeve.

21. The vascular access system of claim 2, wherein the slitted sleeve tip comprises a tip slit, and wherein the tip slit substantially aligns with the longitudinal slit in the slitted sleeve.

22. A vascular access device accessory comprising:
a slitted sleeve;
a slitted sleeve hub, wherein the slitted sleeve is received into a lumen of the slitted sleeve hub; and
a closure;
wherein the slitted sleeve comprises a tubular sleeve body having a longitudinal slit along its length, wherein the slitted sleeve extends from the slitted sleeve hub, wherein the slitted sleeve is adapted to receive a first portion of a medical device therein which is loaded into the slitted sleeve through the longitudinal slit in the slitted sleeve, the tubular sleeve body having a first opening at a proximal end thereof and a second opening at a distal end thereof and a continuous lumen from the first opening to the second opening, the proximal end of the slitted sleeve being received by the lumen of the slitted sleeve hub,
wherein the longitudinal slit closes, but does not seal, after the first portion of the medical device is inserted therein,
wherein the closure is adapted to slide along the slitted sleeve and the first portion of the medical device and comprises:
an outer channel and an inner channel, wherein the outer channel slidably engages the slitted sleeve and the inner channel slidably engages the first portion of the medical device such that sliding the closure along a length of the slitted sleeve draws the first portion of the medical device into the slitted sleeve, and
wherein the slitted sleeve hub comprises a receptacle for receiving and securing the closure therein.

23. The vascular access device accessory of claim 22, wherein the outer channel is at least partially defined by an inner side of an outer wall and an outer side of an inner wall of the closure, and
wherein the inner channel communicates with a radial opening in the closure through which the first portion of the medical device is drawn, wherein the inner channel is at least partially defined by an inner side of the inner wall of the closure.

24. The vascular access device accessory of claim 22, wherein the inner wall of the closure has two portions, each of which extends from a side of the radial opening, each portion extending between the first channel and the second channel, wherein the two portions are not connected.

25. The vascular access device accessory of claim 24, wherein the radial opening has a first surface extending from an outer surface of the outer wall to the inner surface of the inner wall and wherein each inner wall has an inner surface that forms an annular surface in the annular opening.

26. The vascular access device accessory of claim 24, wherein the radial opening is formed in the outer wall and extends therethrough and wherein each of the two portions of the inner wall extend from opposite sides of the radial opening and between the first channel and the second channel, wherein the two portions are not connected.

27. The vascular access device accessory of claim 26, wherein each inner wall has a first inner surface that forms an annular surface in the annular opening and a second inner surface that partially separates the first channel and the second channel.

28. The vascular access device accessory of claim 27 wherein the second inner surface of each of the two portions of the inner wall are curvilinear.

29. The vascular access device accessory of claim 22, further comprising a sealing portion of the slitted sleeve configured to fill a gap and form a seal between an inner surface of a first sheath and a portion of the first portion of the medical device disposed in the first sheath when the first sheath is inserted in a blood vessel and the slitted sleeve is inserted into a first sheath lumen over the portion of the first portion of the medical device disposed in the first sheath, such that, in operation, blood from the blood vessel is substantially prevented from migrating past the seal.

30. The vascular access device accessory of claim 29, wherein the sealing portion of the slitted sleeve is a slitted sleeve tip at the distal open end of the tubular sleeve body, the slitted sleeve tip having an inner surface defining a slitted sleeve tip lumen that is in fluid communication with the slitted tubular sleeve body.

31. A method for inserting a vascular access device accessory into a first sheath, the method comprising the steps of:
inserting a first sheath into the blood vessel through which a medical device is passed, wherein the first sheath has a first sheath lumen extending between a proximal end and a distal end of the first sheath and wherein the medical device has a first portion with a first radial cross section and a second portion with a second radial cross section that is larger than the first radial cross section and wherein the cross section of the first sheath is larger than the first radial cross section;
advancing the second portion of the medical device with the second cross section at least partially through the first sheath;
inserting a portion of the first portion of the medical device into a vascular access device accessory,
wherein the vascular access device accessory comprises:
a slitted sleeve comprising a tubular sleeve body having a longitudinal slit along its length and a first opening at a proximal end of the tubular sleeve body and a second opening at a distal end of the tubular sleeve body and a continuous lumen from the first opening to the second opening;
a slitted sleeve hub, wherein the proximal end of the slitted sleeve is received into a lumen of the slitted sleeve hub and wherein the slitted sleeve extends from the slitted sleeve hub; and
a closure, comprising an outer channel and an inner channel, wherein the outer channel slidably engages the slitted sleeve;
wherein a portion of the first portion of the medical device is inserted into the vascular device accessory by:
placing the first portion of the medical device adjacent to the longitudinal slit of the slitted sleeve;
placing a portion of the first portion of the medical device into the inner channel of the closure;
sliding the closure along at least a portion of the length of the slitted sleeve thereby drawing the first portion of the medical device into the tubular sleeve body of the slitted sleeve through the longitudinal slit in the slitted sleeve; and
advancing the vascular access device accessory over the first portion of the medical device and into a proximal end of the first sheath lumen.

32. The method of claim 31, wherein inserting a portion of the first portion of the medical device into the vascular access device accessory further comprises receiving the closure into the slitted sleeve hub to secure the closure.

33. The method of claim 31, further comprising advancing the slitted sleeve hub into a lumen of a first sheath hub.

34. The method of claim 33, wherein advancing the slitted sleeve hub into the lumen of the first sheath hub comprises coupling the slitted sleeve hub into the first sheath hub via at least one of a threaded connection, a press fit connection, a cliplock connection, locking pin, a clamp, a twist lock, a pop lock, or a snapping fit.
